(19) United States
(12) Patent Application Publication
(10) Pub. No.: US 2002/0069877 A1
(43) Pub. Date: Jun. 13, 2002

(54) Ventilation transport device

(76) Inventors: Daniel C. Villareal, West Fullerton, CA (US); Cynthia Smith, Costa Mesa, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/737,177

(22) Filed: Dec. 13, 2000

(51) Int. Cl. A61M 15/00; A61M 16/10; A62B 7/00; A61M 16/00

(52) U.S. Cl. 128/204.18; 128/203.12

(57) ABSTRACT

A compact, portable transport system enables the application of high-frequency ventilation and inhaled nitric oxide therapy while providing real-time monitoring of the physiological state of the patient. The system includes a patient transport carrier having a patient chamber and an oxygen supply unit, as well as a high-frequency ventilator and a physiologic monitor. An inhaled nitric oxide delivery unit can also be included in the transport system. The nitric oxide system permits the reduction of pulmonary arterial blood pressure, with consequent improvement of patient oxygenation and reduced mortality and morbidity.

### BACKGROUND OF THE INVENTION

1. Field of the Invention

The present invention relates to patient transport and mechanical ventilation systems, and especially to a portable, high-frequency ventilation system.

2. Description of the Related Art

Patients, especially neonates born prematurely, may experience respiratory failure as a result of a number of conditions. These include, among others, respiratory distress syndrome (RDS) due to fetal lung immaturity and consequent lack of surfactant; persistent pulmonary hypertension of the newborn; meconium aspiration syndrome; pulmonary interstitial emphysema; pneumothorax, including tension pneumothorax; pulmonary hypoplasia; congenital diaphragmatic hernia; bronchopulmonary or tracheo-esophageal fistulas; severe pneumonia; and septic shock. Such patients commonly experience severe hypoxemia, which may be caused by such physiologic factors as arteriovenous shunting or ventilation/perfusion (V/Q) mismatching. Such conditions are frequently acute and, unless the patient receives effective treatment within a short period of time, are likely to rapidly worsen, frequently resulting in the death of the patient.

Various strategies have been employed for the treatment of these conditions. These include, among others, conventional mechanical ventilation or intermittent mandatory ventilation, extracorporeal membrane oxygenation, high-frequency ventilation, and the inhalation of pulmonary vasodilators, such as nitric oxide (NO).

Conventional mechanical ventilation has proven to be a less than satisfactory treatment for these illnesses for a number of reasons. Because of the large intra-airway pressures required to oxygenate the patient adequately, there is a great risk of lung damage as a result of barotrauma when conventional ventilation is employed for pediatric patients. Additionally, when infants are maintained at a fraction of inspired O₂ (FIO₂) of 100% at typical high ventilation airway pressures for longer than 24 hours, blindness may result due to retrolental fibroplasia (RLF). Furthermore, the conventional ventilation of such patients is associated with a high rate of granulocyte-related lung injury, as well as the development of air leak syndrome, hyaline membrane disease, and alveolar proteinaceous edema.

Extracorporeal membrane oxygenation has also been employed in treating instances of neonatal respiratory failure. This method requires the presence of an external membrane oxygenator, and its use is generally restricted to well-equipped intensive care units. Although effective, it is often difficult to wean patients from this type of ventilation even after the time for pulmonary recovery has elapsed. It is not a therapy that is well suited to the emergency treatment of pediatric patients experiencing respiratory failure who are located in a facility lacking an intensive care unit.

A third ventilation option is provided by high-frequency ventilation, which is a type of mechanical ventilation. High-frequency ventilation typically employs much lower tidal volumes and a much higher rate of respiration; typically, frequencies of greater than 10 breaths per second are used. High-frequency ventilation represents an improvement over conventional mechanical ventilation in cases of respiratory failure for a number of reasons. Among these are a reduced risk of barotrauma (i.e., pneumothorax, pneumoperitoneum, pneumomediastinum, and subcutaneous emphysema), improved ventilation/perfusion (V/Q) matching, and a lower rate of systemic hypotension. However, the equipment necessary to carry out high-frequency ventilation has hitherto been available only in well-equipped intensive care units. As a result, high-frequency ventilation techniques could not be utilized in patients at remote locations, nor during the transport from such locations to a hospital facility having an intensive care unit equipped to perform high-frequency ventilation.

A fourth method of treatment for these respiratory conditions employs pulmonary arterial vasodilators, such as inhaled nitric oxide (NO). Extensive investigations of the use of nitric oxide as a method of treatment for hypoxemia have been performed. The administration of inhaled nitric oxide induces, in many patients, an improvement in V/Q matching, and it has been postulated that nitric oxide may have benefits unrelated to improved V/Q matching, including anti-inflammatory properties, anti-platelet activity, and effects which diminish vascular permeability. Nitric oxide may be used with conventional mechanical ventilation, but its use has been shown to be particularly effective when combined with high-frequency ventilation; for example, such results were reported by Kinsella, et al., in J. Pediatrics 1995; 126:853-864. As is the case with high-frequency ventilation, however, inhaled nitric oxide therapy is generally available only at hospitals that offer specialized care for critically ill infants. Thus, for children, the most effective therapies for acute respiratory failure have been available almost exclusively in pediatric intensive care units.

However, by their very nature these ailments are often acute and may strike when a child is many miles from a suitably equipped intensive care unit. In addition, the most effective therapies, such as high-frequency ventilation and nitric oxide therapy, are of relatively recent origin. Although they are well-known in academic teaching hospitals, these therapies frequently are not available in the clinics and regional hospitals to which such pediatric patients are often first brought. Typically, the staff of such local facilities is not trained in the use of these techniques, and even if the equipment becomes available, the technological learning curve thereof is quite steep. Therefore, it is desirable, and often mandatory, to transfer such patients to a facility in which these therapies are available and to provide these patients with the optimal therapy as soon as possible.

As noted above, the use of such therapies while transporting a patient to a suitably equipped intensive care unit has hitherto been impossible. The necessary equipment is in the form of separate units, such as a stand-alone high-frequency ventilator, separate inhaled nitric oxide system, and the like, and is extremely bulky. Such equipment would be used in addition to the standard equipment necessary for monitoring and maintaining care to a patient during transport. Therefore, not only would it be quite burdensome to move each piece of equipment along with the patient during transport, due to the limited amount of space in a helicopter or ambulance for transporting the patient, the many pieces of equipment are not likely to fit into the transport vehicle.

### SUMMARY OF THE INVENTION

The inventors recognized the need for a way to, in effect, bring the intensive care unit to the patient in the field. Rather than waiting for the arrival of the patient at a well-equipped intensive care unit to begin optimal therapy, it is much more desirable that the patient begin physiological monitoring, high-frequency ventilation, and, in some cases, inhaled nitric oxide therapy immediately and continue this therapy while en route to an intensive care unit. This therapy provides better oxygenation for the critically ill patient, thereby reducing morbidity and improving survival.

An integrated transport unit in accordance with the present invention permits this type of therapy for children during transport. A compact, portable transport system enables the application of high-frequency ventilation and inhaled nitric oxide therapy, while providing sophisticated monitoring of the physiological state of the patient, such as the assessment of blood oxygen saturation. In one embodiment of the present invention, the transport system incorporates commercially available component units, which are assembled to produce an infant or pediatric transport system equipped with a monitoring system and a high-frequency ventilator. Additionally, the system may incorporate an inhaled nitric oxide system.

Further features and advantages of the present invention will become apparent to one of skill in the art in view of the Detailed Description that follows, when considered together with the attached drawings and claims.

### BRIEF DESCRIPTION OF THE DRAWINGS

FIG. 1 is an oblique front view of a ventilation transport system in accordance with the invention.

FIG. 2 is an exploded front view of the ventilation transport system shown in FIG. 1.

FIG. 3 is an exploded side view of the ventilation transport system shown in FIG. 1.

FIG. 4 is a side cross-sectional view of a ventilation Y tube in an open state.

FIG. 5 is a side cross-sectional view of the ventilation Y tube in a closed state.

FIG. 6 is a front view of a portable pediatric ventilation transport system in accordance with another embodiment of the invention.

### DETAILED DESCRIPTION OF PREFERRED EMBODIMENTS

While the ventilation transport system of the present invention is described herein primarily with respect to pediatric patients, the system can be equally well-suited for use in the adult population. Currently, however, children constitute the most studied group that has been shown to benefit from nitric oxide administration during mechanical ventilation for respiratory failure.

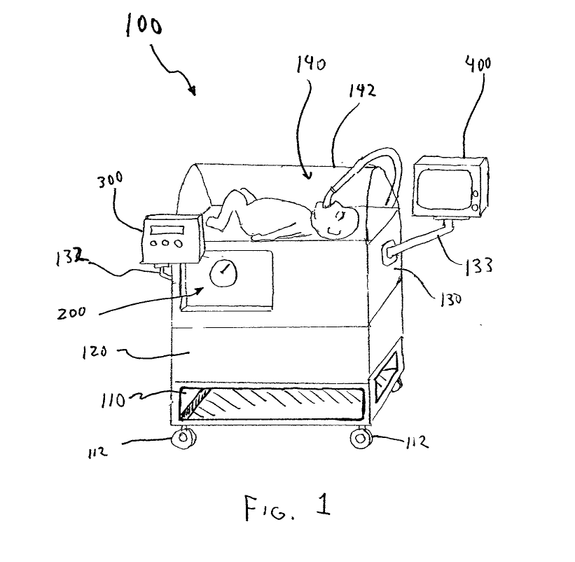

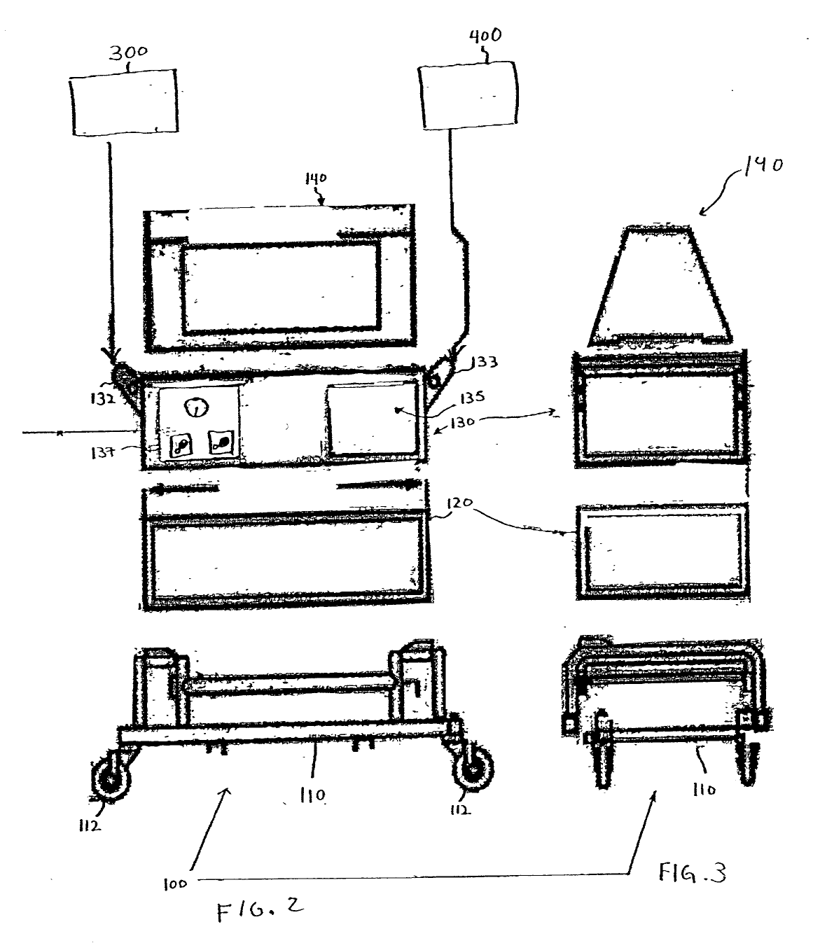

FIGS. 1 through 3 illustrate a ventilation transport system 100 of the present invention adapted for the transport of infants or small children. FIG. 1 shows an oblique schematic view of the portable ventilation transport system of the present embodiment. FIG. 2 is an exploded frontal view of the ventilation transport system and FIG. 3 is an exploded side view of the portable ventilation transport system of the present embodiment. The ventilation transport system 100 includes a mechanical or high-frequency ventilator 200 installed in a main body 130, a physiological monitor 400 attached to the main body 130, and an inhaled nitric oxide delivery system 300, also attached to the main body 130. The physiological monitor 400 and the inhaled nitric oxide delivery system 300 are attached to the main body 130 by brackets. The transport main body 130 includes a respiratory alcove 135 and a ventilator alcove 137. The respiratory alcove 135 serves to store supplies used in treating patients during transport. The ventilator alcove 137 houses the ventilator 200. Commercially available unit components are assembled to form the remainder of the transport system of the first embodiment. In the illustrated infant ventilation transport system 100, these are an oxygen supply chamber 110, an accessory module 120, a transport main body 130, and a patient chamber 140. The accessory module 120 is used with other modifications such as further monitors, sensors, intensive care devices, and the like. These component units will be described in greater detail hereinbelow.

In the case of treating babies with respiratory failure, the mechanical ventilator 200 is preferably a high-frequency ventilator, and more preferably a high-frequency ventilator which is capable of conducting at least one of the following ventilatory modes: positive pressure high-frequency ventilation, high-frequency jet ventilation, and high-frequency oscillatory ventilation. As shown in FIG. 2, the ventilator 200 is located within the ventilator alcove 137 of the transport main body 130. In one embodiment, the ventilator 200 is a “PERCUSSION AIR DUOTRON” high-frequency ventilator. Alternatively, a “VDR®-3C Universal Logistical Percussionator®” produced by the Percussionaire® Corporation (Sandpoint, Ind.) can be used as the high frequency ventilator 200. These are, however, simply examples, and the skilled artisan will readily appreciate that other ventilation devices may be employed without thereby departing from the spirit and scope of the present invention.

In order to provide the benefits of inhaled nitric oxide therapy, the pediatric ventilation transport system 100 further includes a nitric oxide delivery system 300. This nitric oxide delivery system 300 is secured to the transport main body 130 by a bracket 132, which may be provided on the transport main body 130. This nitric oxide delivery system 300 is preferably capable of delivering therapeutic doses of inhaled nitric oxide, and advantageously permits monitoring of NO and NO₂ levels in the patient. This monitoring may advantageously employ a mass flow meter. The “AeroNOx Portable System” produced by Pulmonox Medical Corporation (Alberta, Canada) is one example of a nitric oxide delivery system 300 which may be used in the ventilation transport system 100. This is only an example, however, and the skilled artisan will readily appreciate that other nitric oxide systems may be employed without thereby departing from the spirit and scope of the present invention.

In the illustrated embodiment, the nitric oxide delivery system 300 is attached to the main body 130 by brackets. In other embodiments, however, the nitric oxide delivery system can be integral with the ventilation transport system 100.

As described above, the nitric oxide delivery system 300 of the present embodiment is capable of monitoring NO and NO₂ levels. The nitric oxide delivery system 300 may employ either a chemiluminescence or an electrochemical detection method. Using the “AeroNOx Portable System” described above, the present inventors have experimentally determined that the electrochemical method exhibits negligible differences from the chemiluminescence method, although the latter is considered the “gold standard” in the industry. Thus, either the chemiluminescence or the electrochemical detection method for NO and NO₂ gases may be employed in the ventilation transport system 100.

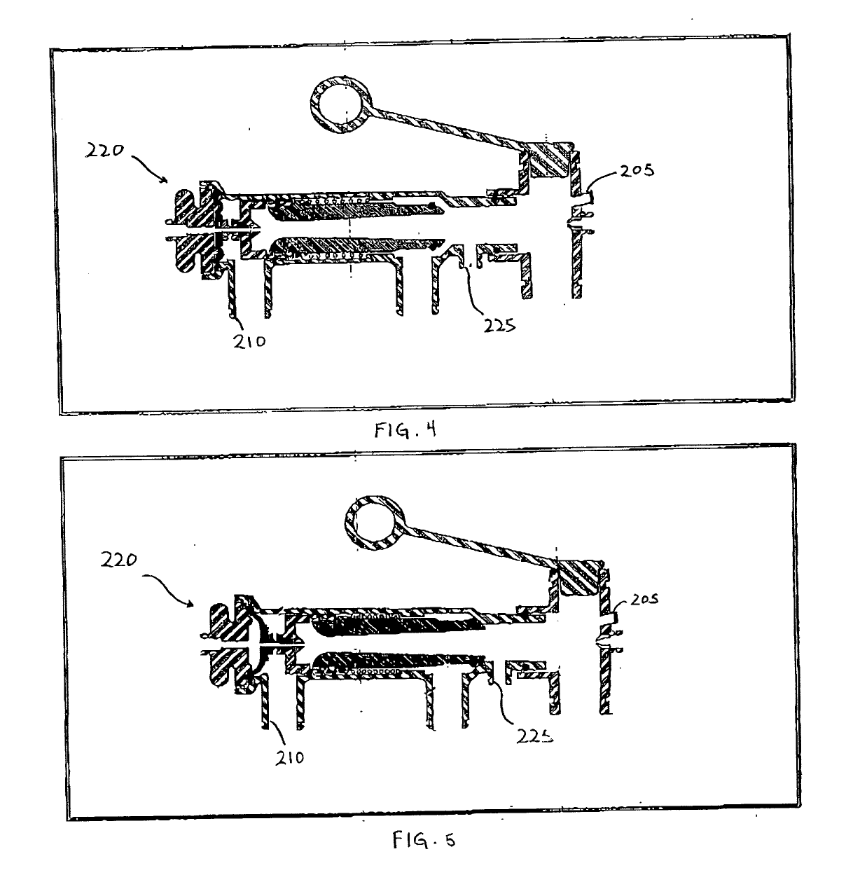

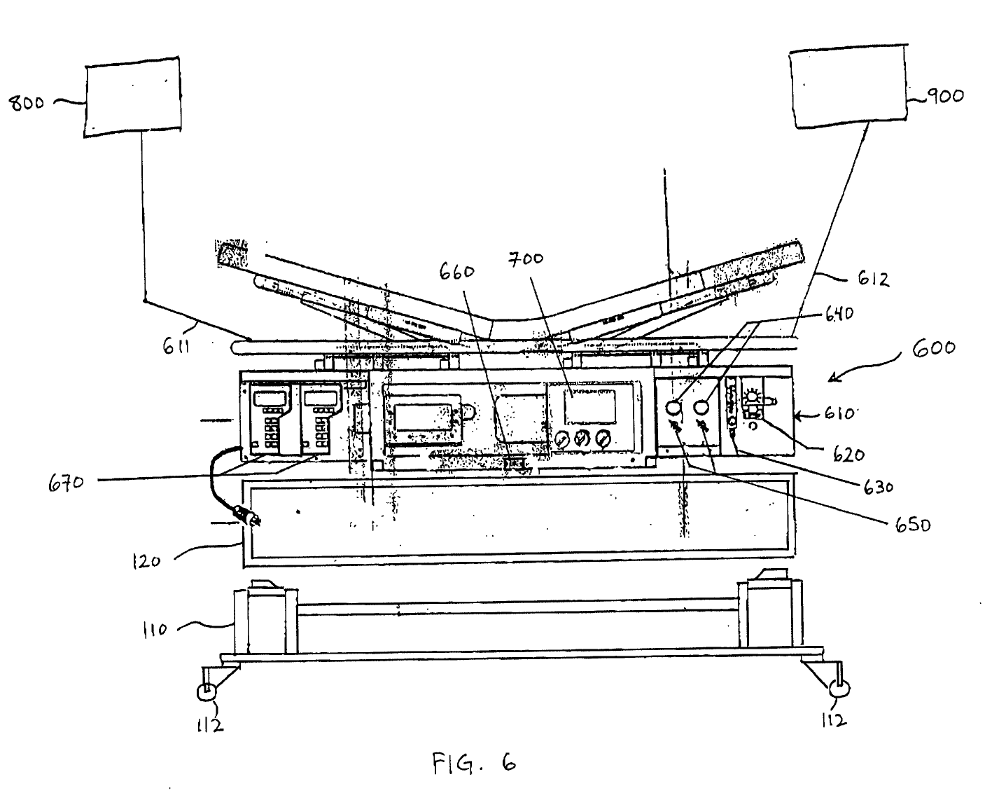

FIGS. 4 and 5 illustrate a ventilation tube 220 of the ventilation transport system 100. The ventilator tubing facilitates the sampling of nitric oxide at a point downstream from the instillation point 225 of the nitric oxide (NO) gas, as shown in FIGS. 4 and 5. FIG. 4 depicts a Phasitron Y tube, which is a standard sliding Venturi valve system. This Y tube is modified by adding a port 205 for nitric oxide sampling. As shown in FIGS. 4 and 5, this port 205 is provided at a point downstream from the entrainment port 210 of the Y tube so as to permit NO gas sampling. This permits an operator to determine whether gas mixing is adequate. FIG. 4 depicts the state in which the valve system is unpressurized and open for passive expiratory flow, while FIG. 5 depicts the state in which the valve system is pressurized and closed for inspiratory subtidal-volume injection.

The portable pediatric ventilation transport system 100 also includes a physiological monitor 400 attached to the ventilation transport system 100. In the illustrated embodiment, the physiological monitor 400 is mounted to the transport main body 130 by a bracket 133. This physiological monitor 400 is of a standard battery-driven type, and is capable of monitoring various patient parameters, including respiration, SpO₂ (pulse oximetry), ECG, non-invasive blood pressure, invasive blood pressure, carbon dioxide (end-tidal CO₂), and nitrous oxide. One example of a physiological monitor 400 is the “Millennia Patient Monitor,” produced by Invivo Research Incorporated (Orlando, Fla.). This again is simply one example, and the skilled artisan will appreciate that other physiological monitors may be employed without departing from the spirit and scope of the present invention.

The oxygen supply chamber 110 provides storage for gas (oxygen) cylinders. Wheels 112 are attached to the ventilation transport system 100 at oxygen supply chamber 110.

The oxygen supply chamber 110 may accommodate a plurality of gas cylinders to enable sufficient oxygen administration during transport, as shown, for example, in the system and method disclosed in U.S. patent application Ser. No. 09/405,316, which is incorporated in its entirety herein by reference. In order to allow high-flow, high-pressure oxygen tanks to be adapted to a portable ventilator and to allow sufficient oxygen to be administered throughout the course of prolonged transport of a patient, the oxygen delivery system (not illustrated) may include at least a first set and a second set of individual oxygen tanks. A first intake tube is interposed between the first set of oxygen tanks and a first regulator, and the first regulator contains a valve that remains open until the pressure of oxygen flowing through the first regulator drops below a predetermined threshold pressure level. A second intake tube is interposed between the second set of oxygen tanks and a second regulator, and the second regulator contains a valve which remains closed until the pressure in the second regulator drops to approximately the predetermined threshold pressure level. This threshold pressure level is, in some embodiments, within the range of 90 to 100 pounds per square inch. One or more outtake tubes connect the first and second regulators, and a central tube is interposed between these outtake tubes and a mechanical ventilator. In some embodiments, there are two sets of oxygen tanks. One or more pressure gauges may be attached to the regulators (not illustrated). In addition, other supplies can also be stored in the oxygen supply chamber 110.

The illustrated patient chamber 140 is of a type that is commonly used in intensive care units and, in the case of chambers or beds sized for infants, may include a warming device (not shown) to regulate the temperature of the environment in which the patient is transported. This assists in maintaining the patient's body temperature at a normal level during transport. The patient chamber 140 illustrated in FIGS. 1-3 has a bed adapted for infant use and is provided with a cover 142 that is, at least partially, substantially transparent. Although not illustrated, the cover 142 may include a head access door, two hand insertion ports, and accessory ports for IV or respiratory tubing.

One example of a patient chamber 140 that can be used in the ventilation transport system 100 is the “Multipurpose Infant Transport System Model 20H” produced by International Biomedical, Inc. (Austin, Tex.), which includes an incubator with a double-wall hood, a head access door, a front access door with two hand insertion ports, accessory ports for IV and respiratory tubing, a mattress, a high-intensity exam light, a skin temperature probe, an accessory module, an oxygen cart module, a double-wall hood for pediatric transport, IV syringe pumps, a humidifier, a suction device, a blender, and oxygen analyzer, a ventilator monitor, a physiological monitor and an infant ventilator. The “Multipurpose Infant Transport System Model 20H” does not include a high-frequency ventilator or a nitric oxide delivery system, and its physiological monitor cannot measure certain parameters measured by the physiological monitor 400 of the ventilation transport system 100, such as nitrous oxide.

An advantage of the ventilation transport system 100 is its compactness. Typically, the transverse width of the system ranges from about 15 inches to about 35 inches. In some embodiments, the transverse width of the device, including the cart, bed, and monitor, does not exceed 30 inches. In other embodiments, the width of the device does not exceed 25 inches. This relatively narrow width allows the ventilation transport system to be easily moved into and out of ambulances, emergency transport helicopters, and other patient transport vehicles, thus making it possible to conduct high frequency ventilation and inhaled nitric oxide therapy while a patient is being transported. As discussed above, due to space limitations within the transport vehicle, this was not previously conventionally possible. To accomplish this, the height and width of the device are adapted to fit within the patient-entry doorway of an ambulance or transport helicopter. For example, a year-2000 Ford model F350 ambulance, such as those operated by Lynch Ambulance Company (Anaheim, Calif.), has rear doors which open to form a patient-entry space of about 55 inches in height and 46 inches in width. Thus, the width of the ventilation transport system 100, that is to say, the distance in FIG. 3 from the left side of the system to the right side of the system, allows for easy entry into this type of standard ambulance for patient transport. Other exemplary dimensions for the ventilation transport system 100 are as follows: height (from bottom to top of the system in FIG. 2): about 35 to about 50 inches; and length (from left side to right side of the system in FIG. 2): about 34 to about 50 inches.

Furthermore, a second embodiment of the present invention is depicted in FIG. 6; this figure shows an embodiment adapted for the transport of persons larger than infants. The pediatric ventilation transport system 600 of this embodiment is similar to the ventilation transport system 100 described in connection with FIGS. 1-3, except that it is modified to accommodate larger children or small adults.

This transport system is provided with a mechanical or high-frequency ventilator 700 installed in a support module 610, a physiological monitor 900, and an inhaled nitric oxide delivery system 800. The physiological monitor 900 and the inhaled nitric oxide delivery system 800 are attached to the support module 610 via brackets 611 and 612. Commercially available unit components are assembled to form the remainder of the transport system of the second embodiment.

As in the first embodiment above, the pediatric transport system 600 of this embodiment is provided with a ventilator 700, which is preferably a mechanical ventilator, and more preferably a high-frequency ventilator. Advantageously, the high-frequency ventilator is capable of ventilating the patient in at least one of the following modes: positive pressure high-frequency ventilation, high-frequency jet ventilation, and high-frequency oscillatory ventilation. In one embodiment, the “VDR®-3C Universal Logistical Percussionator®,” produced by the Percussionaire® Corporation (Sandpoint, Ind.), was employed as this ventilator 700. Other ventilators may be employed, as will be apparent to those of skill in the art.

The pediatric ventilation transport system 600 also includes a nitric oxide delivery system 800, which is mounted on bracket 611 of the support module 610. As in the first embodiment described above, this nitric oxide delivery system 800 is preferably a nitric oxide delivery system capable of delivering therapeutic doses of inhaled nitric oxide. Advantageously, the nitric oxide delivery system is capable of monitoring of NO and NO₂ levels, and may employ a mass flow meter. As in the embodiment described above, either the chemiluminescence or the electrochemical detection method for NO and NO₂ gases may be employed. In the present embodiment, the “AeroNOx Portable System” produced by Pulmonox Medical Corporation (Alberta, Canada) was employed. The skilled artisan will readily appreciate that other nitric oxide systems may be employed and are within the scope of the invention.

In this embodiment of the pediatric ventilation transport system 600, as in the embodiment described above, a further modification is made to the ventilator tubing 220 in order to facilitate the sampling of nitric oxide at a point downstream from the instillation point of the NO gas, as shown in FIGS. 4 and 5. As shown in FIG. 3, the Phasitron Y tube, which is a standard sliding Venturi valve system, is modified by the addition of a port 205 for nitric oxide sampling. As shown, this port is provided at a point downstream from the entrainment port 210 of the Y tube so as to permit NO gas sampling. This permits an operator to determine whether gas mixing is adequate. FIG. 4 depicts the state in which the valve system is unpressurized and open for passive expiratory flow, while FIG. 5 depicts the state in which the valve system is pressurized and closed for inspiratory subtidal-volume injection.

In addition, the pediatric ventilation transport system 600 of the present embodiment is further provided with a physiological monitor 900, which is mounted to a bracket 612 of the support module 610. As in the first embodiment described above, this physiological monitor 900 is preferably of a standard battery-driven type because it may be used in the field, away from a wall source of electricity.

In addition, a number of commercially available component units are assembled with the ventilator 700, the nitric oxide delivery system 800, and the physiological monitor 900 to produce the pediatric ventilation transport system 600 of the present embodiment. The bed 613 thereof is adapted for persons larger than infants. The pediatric ventilation transport system 600 includes a support module 610, a blender 620, a flow meter 630, gas contents gauges 640, shut off valves 650, an oxygen cart latch 660, and syringe pumps 670. These component units are representative of those commonly employed in conventionally available pediatric transport systems.

The pediatric transport system 600 also includes a folding cart 710, or an oxygen tank chamber that is provided with wheels to facilitate transport, as is depicted in FIG. 6. As in the embodiment described above, the oxygen tank cart 710 may accommodate a plurality of gas cylinders to enable sufficient oxygen administration during transport, as shown, for example, in the system and method disclosed in U.S. patent application Ser. No. 09/405,316. In this embodiment, a pediatric transport cart that was employed was the “Pedi-Porter” pediatric transport cart produced by International Biomedical, Inc. (Austin, Tex.). The “Pedi-Porter” is adapted for use with persons who are larger than infants. The “Pedi-Porter” pediatric transport cart includes a stretcher, a support module with yokes and regulators for gas cylinders, gas contents gauges, shut off valves, external gas connections, two IV poles, a mattress, a patient restraint system, a blender, a flowmeter, IV syringe pumps, a ventilator, and a physiological monitor. The “PediPorter” pediatric transport system does not include the high frequency ventilator or nitric oxide delivery system of the ventilator transport system 100, and its physiological monitor cannot measure certain parameters measured by the physiological monitor 900 of the ventilation transport system 100, such as nitrous oxide. Other pediatric transport carts can also be employed in the pediatric ventilation transport system 600.

Use of the ventilation transport systems 100 and 600 will now be described in detail. After an incident of respiratory failure is reported in a remote location, a transport team may be dispatched by air or ground ambulance, together with the portable ventilation transport system 100 or 600, depending on the size of the patient. On arrival, the patient is placed on or in the transport system and is connected to the physiological monitor via ventilator tubing 220. Mechanical ventilation and, if necessary, nitric oxide administration are initiated. Often, a neuromuscular blocking agent such as pancuronium bromide (Pavulon™) or succinylcholine chloride (Anectine™) is administered to the patient, along with a sedative, to facilitate ventilation. Once this has been accomplished, the patient is transported on or within the transport system to a hospital intensive care unit, where therapy may be continued.

From the foregoing description, it will be appreciated that a novel approach for the ventilatory transport of patients has been disclosed. While aspects of the invention have been described with reference to specific embodiments, the description is illustrative and is not intended to limit the scope of the invention. Various modifications and applications of the invention may occur to those who are skilled in the art, without departing from the true spirit or scope of the invention. The breadth and scope of the invention should be defined only in accordance with the appended claims and their equivalents.

What is claimed is:

1. A ventilation transport device, comprising:
a portable cart having a top and sides adapted to facilitate propulsion of the portable cart by human beings;
a patient chamber adapted to accommodate a patient, the patient chamber being attached to the portable cart; and
a high-frequency ventilator adapted to conduct high-frequency ventilation of the patient, the high-frequency ventilator being connected to the portable cart.

2. A device in accordance with claim 1, wherein the high-frequency ventilator is capable of performing ventilation in at least one mode selected from the group consisting of high-frequency jet ventilation, high-frequency positive pressure ventilation, and high-frequency oscillatory ventilation.

3. A device in accordance with claim 1, wherein the ventilation transport device has a height and width, the height and width each being less than the height and width of a patient-entry doorway of an ambulance.

4. A device in accordance with claim 1, further comprising a monitor attached to the portable cart.

5. A device in accordance with claim 4, wherein the monitor is capable of monitoring at least one parameter selected from the group consisting of nitric oxide concentration, heart rate, arterial oxygen saturation, an electrocardiogram, systemic arterial blood pressure, pulmonary arterial pressure, central venous pressure, and end-tidal CO₂.

6. A device in accordance with claim 1, further comprising a nitric oxide delivery system attached to the portable cart.

7. A device in accordance with claim 6, wherein the nitric oxide delivery system further comprises a mass flow meter.

8. A device in accordance with claim 6, wherein a width of the device does not exceed 30 inches, a length of the device does not exceed 45 inches, and a height of the device does not exceed 50 inches.

9. A device in accordance with claim 8, wherein a width of the device does not exceed 25 inches, a length of the device does not exceed 40 inches, and a height of the device does not exceed 45 inches.

10. A device in accordance with claim 1, wherein the patient chamber is sized for infant use.

11. A device in accordance with claim 10, further comprising a warming apparatus connected to the portable cart.

12. A device in accordance with claim 11, further comprising a cover connected to the portable cart, the cover comprising a portion that is substantially transparent.

13. A device in accordance with claim 12, wherein the cover comprises at least one accessory port for tubing.

14. A device in accordance with claim 1, wherein the patient chamber is adapted for persons larger than infants.

15. A device in accordance with claim 1, wherein the portable cart includes an oxygen supply chamber.

16. A transport device adapted to facilitate propulsion of the transport device by human beings, comprising:
a patient chamber adapted to accommodate and secure a patient during transport;
a mechanical ventilator adapted to conduct mechanical ventilation of the patient, the mechanical ventilator being coupled to the patient chamber, and
a nitric oxide delivery system adapted to provide the patient with inhaled nitric oxide therapy, the nitric oxide delivery system being coupled to the patient chamber.

17. A device in accordance with claim 16, wherein the nitric oxide delivery system further comprises a mass flow meter.

18. A device in accordance with claim 16, wherein the patient chamber is sized for infant use.

19. A device in accordance with claim 16, further comprising a warming apparatus connected to the patient chamber.

20. A device in accordance with claim 18, further comprising a cover connected to the patient chamber, the cover comprising a portion that is substantially transparent.

21. A device in accordance with claim 20, wherein the cover is provided with at least one accessory port for tubing.

22. A device in accordance with claim 16, wherein the patient chamber is adapted for persons larger than infants.

23. A device in accordance with claim 16, further comprising a physiological monitor coupled to the patient chamber.

24. A device in accordance with claim 23, wherein the monitor is capable of monitoring at least one parameter selected from the group consisting of nitric oxide concentration, heart rate, arterial oxygen saturation, an electrocardiogram, systemic arterial blood pressure, pulmonary arterial pressure, central venous pressure, and end-tidal CO₂.

25. A device in accordance with claim 16, further comprising an oxygen supply device coupled to the patient chamber.

26. A device in accordance with claim 25, wherein the oxygen supply device includes at least one oxygen tank.

27. A transport cart adapted to facilitate propulsion of the transport cart by human beings while providing respiratory support to a patient, the transport cart comprising:
a patient chamber adapted to accommodate the patient;
a high-frequency ventilator connected to the patient chamber;
a nitric oxide delivery system connected to the patient chamber;
a monitor capable of monitoring at least one parameter selected from the group consisting of nitric oxide concentration, heart rate, arterial oxygen saturation, an electrocardiogram, systemic arterial blood pressure, pulmonary arterial pressure, central venous pressure, end-tidal CO₂, and respiratory rate, the monitor being connected to the patient chamber;
a warming apparatus connected to the patient chamber; and
a transparent cover connected to the patient chamber and having at least one port permitting hand insertion.

28. A transport cart as in claim 27, wherein a height and a width of the system are adapted to fit within the patient-entry doorway of an ambulance.

29. A transport cart in accordance with claim 28, wherein a width of the device does not exceed 25 inches, a length of the device does not exceed 40 inches, and a height of the device does not exceed 45 inches.
